(19) United States
(12) Patent Application Publication
(10) Pub. No.: US 2021/0299293 A1
(43) Pub. Date: Sep. 30, 2021

(54) SOLID WASTE TREATMENT CENTER FOR MITIGATION OF INFECTIOUS PATHOGEN SPREAD

(71) Applicant: Ecogensus LLC, Hartford, CT (US)

(72) Inventor: Bjornulf Ostvik, Hartford, CT (US)

(21) Appl. No.: 17/211,305

(22) Filed: Mar. 24, 2021

Related U.S. Application Data
(60) Provisional application No. 62/994,184, filed on Mar. 24, 2020.

(51) Int. Cl. A61L 2/04; A61L 11/00

(52) U.S. Cl. CPC A61L 2/04; A61L 11/00

(57) ABSTRACT

Mixed solid waste is transported from a waste collection vehicle to a first room of a waste treatment facility. The first room defines an enclosed isolation zone that is configured to receive the mixed solid waste from the waste collection vehicle. The mixed solid waste is treated in at least one process vessel in the first room to destroy a majority of pathogens in the mixed solid waste to yield a sanitized waste. The sanitized waste is transported to a second room of the facility. The second room defines a clean zone that is separated from the first room by a barrier.

### CROSS-REFERENCE TO RELATED APPLICATION

This application claims the benefit of U.S. Patent Application Ser. No. 62/994,184 entitled “SOLID WASTE TREATMENT CENTER FOR MITIGATION OF INFECTIOUS PATHOGEN SPREAD” and filed on Mar. 24, 2020, which is incorporated by reference herein in its entirety.

### TECHNICAL FIELD

This disclosure relates generally to solid waste management facilities, such as waste transfer stations, materials recovery facilities, and recycling centers. More particularly, this disclosure relates to improved layouts and design for waste management facilities for mitigation of infectious disease transmission.

### BACKGROUND

Solid waste, including but not limited municipal solid waste, rubbish, garbage, commercial waste, and industrial waste, is a growing infrastructure challenge globally. It has been estimated that the amount of waste generated annually will double in the period 2013-2025. Such a rapid rate of growth already places significant strains on solid waste management infrastructure and, in particular, disposal. The cost of disposal in developed countries, where regulations govern landfills, has been steadily rising for decades. In developing countries, societies are deeming proper solid waste management to be a priority infrastructure improvement objective, especially in light of the public health and environmental risks posed by poor solid waste disposal practices.

### SUMMARY

This disclosure relates to systems and methods for handling and treating solid waste in a solid waste treatment facility designed to mitigate the spread of infectious disease. In some embodiments, the facility includes an enclosed building, divided into zones whereby one zone is designated to handle the material prior to cleaning treatment (e.g., sterilization) and another is designated as a clean, post-treatment zone. The first zone is essentially an isolation zone, with a separate air handling system designed to treat the air within the zone for airborne organisms (as well as other pollutants). Waste trucks enter and exit the isolation zone via one or two intermediate containment areas, first entering through the door to the external environment into the intermediate area, waiting for the first door to close, and then entering or exiting the facility via a second door between the containment area and the isolation zone. Each door is outfitted with an industrial air curtain or similar device. Sorting of waste occurs in the isolation zone, including removing recyclable materials that might be present such as metals, glass and aggregate. With the exception of the aggregate (rocks, sand, dirt), the recyclable materials can be baled in plastic. The aggregate can be removed to a concrete (or similar) bunker and kept covered until it is time for pick up and removal. Such aggregate can be used for construction and road projects. The remaining waste is moved into treatment systems designed with the capability to decontaminate (e.g., sterilize) the waste. In some embodiments, such systems include thermal treatment systems or autoclaves.

Unlike medical waste (e.g., regulated medical waste), which is transported in sealed containers or bags directly to an autoclave or incinerator, a challenge with “non-hazardous” waste is the sheer volume of it and that it is not sealed upon arrival to the facility. Therefore, the post-sorted waste can be transported in the isolation zone (ideally by enclosed conveyor to minimize the need for human personnel inside the isolation zone) and fed (e.g., directly fed) into the thermal treatment systems as a way to mitigate the potential of contamination. An exemplary method for feeding the material includes the use of rotary airlock valves (or similar devices), attached between the transport conveyors and the processing plenum of the treatment systems.

The material can exit the thermal treatment systems through an inner isolation wall and, having been sanitized, can be placed in the clean zone. The waste can be kept in the clean zone until cooled by a cooling system and then transported away from the facility. This treated waste can be landfilled or recycled but is, ideally, used as a fuel due to its high carbon content and heating value, depending on the composition of the waste, especially post-treatment.

In another embodiment, especially suitable for immediate treatment of solid waste, mobile thermal treatment systems are placed within existing waste management facilities or at landfills, in order to allow for sterilization of waste. In this embodiment, there would be greater need for personal protective equipment for human personnel, but it is preferable to the alternative of not sterilizing solid waste.

Certain aspects of the subject matter described can be implemented as a solid waste treatment facility. The facility includes a first room and a second room. The first room defines an isolation zone having at least one access gate (for example, an access gate for entry and an access gate for exit). The at least one access gate is sized to receive a waste collection vehicle. The first room is an enclosed area. The first room is configured to receive mixed solid waste (e.g., non-hazardous waste) from the waste collection vehicle. The first room includes a receiving area and at least one process vessel. The receiving area is configured for receiving the mixed solid waste from the waste collection vehicle. The at least one process vessel is configured to reduce the amount of or eliminate at least a portion of pathogens present in the mixed solid waste to yield sanitized waste that is free of or substantially free of pathogens. The second room defines a clean zone. The second room is separated from the first room by a barrier. The second room is configured to receive the sanitized waste from the first room.

This, and other aspects, can include one or more of the following features.

In some embodiments, the mixed solid waste includes municipal waste. The municipal waste can include waste obtained from a residential source, an institutional source, a commercial source, an agricultural source, a sewage source, or any combination of these. The mixed solid waste can be substantially free of medical waste (e.g., regulated medical waste), for example, originating from a laboratory or dedicated building that provides medical care. In some embodiments, the mixed solid waste is non-hazardous waste.

In some embodiments, the waste collection vehicle includes a garbage truck, a refuse vehicle, a semi-truck, or any transport vehicle that includes a compartment for receiving and transporting waste.

In some embodiments, the first room includes one or more containment areas. The one or more containment areas can be configured to receive the waste collection vehicle. The first room can define a negative air pressure relative to an external environment. The first room can include one or more hydraulic push floors, tipping floors, or any combination of these, which can be configured to move the mixed solid waste from the receiving area to the at least one process vessel, move the sanitized waste from the first room to the second room, or both. The first room can include one or more industrial air curtains at the at least one access gate of the first room. For example, the first room includes an industrial air curtain at an entry of the first room, an exit of the first room, or both.

In some embodiments, the facility includes a heater that is operatively coupled to the at least one process vessel. The at least one process vessel can be configured to receive an amount of the mixed solid waste and increase a temperature of the mixed solid waste to yield the sanitized waste that is free of or substantially free of pathogens.

In some embodiments, the second room includes a cooling system. The cooling system can be configured to cool sanitized waste. The cooling system can be an air cooling system.

Certain aspects of the subject matter described can be implemented as a method of treating solid waste at a waste treatment facility. Mixed solid waste is transported from a waste collection vehicle to a first room of the facility. The first room defines an enclosed isolation zone that is configured to receive the mixed solid waste from the waste collection vehicle. The mixed solid waste is treated in at least one process vessel in the first room to destroy a majority of pathogens in the mixed solid waste to yield a sanitized waste. The sanitized waste is transported to a second room of the facility. The second room defines a clean zone that is separated from the first room by a barrier.

This, and other aspects, can include one or more of the following features.

In some embodiments, the isolation zone of the first room is sealed by applying a negative air pressure relative to an external environment outside the first room and/or the facility.

In some embodiments, treating the mixed solid waste includes heating a majority of the mixed solid waste to a temperature of at least 121° C., at least 132° C., at least 141° C., or at least 160° C. Heating the mixed solid waste can include heating a majority of the mixed solid waste to at least 121° C. for at least 30 minutes or at least 132° C. for at least 4 minutes. In some embodiments, treating the mixed solid waste includes drying the mixed solid waste to yield a dried waste that includes about 5 wt. % or less of water. In some embodiments, treating the mixed solid waste includes exposing the mixed solid waste or the dried waste to negative pressure. In some embodiments, treating the mixed solid waste includes exposing the mixed solid waste or the dried waste to an operating pressure of atmospheric pressure or greater and agitating the mixed solid waste during the heating.

In some embodiments, the method includes sorting the mixed solid waste, removing recyclables from the mixed solid waste and baling the recyclables, shredding the mixed solid waste, or any combination of these.

In some embodiments, transporting the mixed solid waste from the waste collection vehicle includes moving the mixed solid waste, in an unsealed state, onto one or more tipping floors, hydraulic push floors, or any combination of these. Transporting the mixed solid waste from the waste collection vehicle can include feeding the mixed solid waste to the at least one process vessel via a conveyor.

In some embodiments, the mixed solid waste is treated to yield a waste product for landfill allocation. In some embodiments, the mixed solid waste is treated to yield a raw material for a reuse or recycled product.

In some embodiments, the method includes preventing pathogens in the mixed solid waste from entering the second room of the facility by applying positive pressure in the second room, separating the first and second rooms with a barrier, or any combination of these

In some embodiments, the method does not incinerate or autoclave the mixed solid waste to yield the sanitized waste.

In some embodiments, a majority of emissions produced by the method is water vapor.

Certain aspects of the subject matter described can be implemented as a method of treating solid waste at a waste treatment facility. Mixed solid waste is transported from a waste collection vehicle to a first room of the facility. The first room defines an enclosed isolation zone that is configured to receive mixed solid waste from the waste collection vehicle. The mixed solid waste is substantially free of medical waste. The mixed solid waste is treated in at least one process vessel in the first room to destroy a majority of pathogens in the mixed solid waste to yield a sanitized waste. The sanitized waste is transported to a second room of the facility. The second room defines a clean zone separated from the first room by a barrier.

While multiple embodiments are disclosed, still other embodiments of the present disclosure will become apparent to those skilled in the art from the following detailed description, which shows and describe illustrative embodiments of the disclosure. As will be realized, embodiments are capable of modifications in various aspects, all without departing from the present disclosure. Accordingly, the drawings and detailed description are to be regarded as illustrative in nature and not restrictive.

### BRIEF DESCRIPTION OF THE DRAWINGS

FIG. 1A depicts an example waste treatment facility.

FIG. 1B depicts an example thermal treatment system that can be implemented in the waste treatment facility of FIG. 1A.

FIG. 2 is a flow chart of an example method for handling waste.

### DETAILED DESCRIPTION

The “first public health” revolution, generally occurring in the period 1880-1920, saw an increased focus on sanitation matters as a way to mitigate infectious disease spread—one of the primary areas that was addressed in that period was municipal waste collection. The objective, at that time, was simply the removal of the solid waste from immediate urban areas where humans lived (such as removal of solid waste from the streets, regular collection of waste from households, and so forth). Solid waste was carried outside of the urban area in an effort to prevent the waste from contributing to infectious disease spread.

Once waste was brought to landfills just outside of the urban areas, there still remained the risk of disease transmission as well as the spread of other toxic and hazardous substances (such as heavy metals). In the United States, the Solid Waste Disposal Act (SWDA) was passed in 1965 to begin transitioning away from the concept of “dumps” (mere discarding of solid waste into designated outdoor areas) to regulated landfills. At the time, the U.S. Environmental Protection Agency saw the SWDA as the first federal effort to improve solid waste disposal. In 1976, the SWDA was amended and superseded by the Resource Conservation and Recovery Act (RCRA), which remains the governing law in the United States today. RCRA addressed principally solid waste, hazardous wastes and underground storage tanks. As it pertains to Solid Waste, RCRA tasked the states with developing criteria for solid waste landfills and prohibiting open dumping. Similar laws developed throughout the world, though much of the world has significant challenges with open dumping today.

Two major shortcomings of the paradigm established by laws such as RCRA (and the infrastructure that flowed from the paradigm) was (1) the potential for infectious disease transmission caused by the waste (and its transport) on its journey to the landfill and (2) the remaining potential for landfills to spread infectious disease.

There are numerous mechanisms by which solid waste remains a major contributor to infectious disease spread. These diseases include Coronaviridae (which includes human coronaviruses), Staphylococcaceae, Enterobacteriaceae (which includes E. Coli and Salmonella), and even microorganisms commonly thought of as only causing human-to-human disease transmission, such as Picornaviridae (which includes Hepatovirus that causes Hepatitis A). The potential mechanisms include: (1) contact between municipal waste workers and the solid waste (transmission from fomites as well as even potential aerosolization of organisms during waste handling), (2) transmission by birds and other animals of zoonotic diseases, (3) vector transmission by anthropods (including mosquitoes, mites, fleas and ticks), (4) leaching of organisms from waste into the environment in a manner where the organisms are transmitted to animals or humans. In the solid waste collections operations that currently exist, all four mechanisms cited above can occur.

Solid waste management practices, including even modern landfilling approaches, contribute to disease spread and also increase the risk of zoonotic pathogens, including new mutated variants, emerging to threaten human health. Solid wastes, including landfills, provide a food source, breeding sites, and animal dwellings for disease vectors—including rodents, insects, avian vectors, bats, and various mammals. In addition, during times of disease outbreaks (either natural, unintentional releases, or due to bioterrorism), landfills may pose a role in continuation of disease spread. For example, in 2018 the U.S. Environmental Protection Agency reported that viruses can persist for days to months in landfill leachate. The EPA stated that “results from this study suggest that in the event of a significant adversarial attack on the U.S. population using viral BW agents, an unintentional release, or a natural outbreak (e.g., Zika virus, Ebola virus) where biological waste is disposed of without adequate decontamination, there can be a potential threat to human health and the environment if the virus reaches the landfill leachate.” There is a lack of protocol, processes, and systems tailored to decontaminating municipal solid waste (e.g., garbage or rubbish) before landfill disposal. Indeed, only small batches of specific wastes directly generated in health care or laboratory settings are decontaminated. Those wastes are typically decontaminated by autoclave or incineration, in which the former can be impractical and too costly for treatment of the hundreds of millions of tons of municipal solid waste produced each year, and the latter can be unfavorable due to high costs and negative environmental impact (e.g., contribution to the production of additional greenhouse gases).

This disclosure describes a facility and a method for managing and treating (e.g., decontaminating, sanitizing, and/or sterilizing) solid waste coming from the community (residential, commercial, and industrial settings). This solid waste is commonly referred to as “municipal solid waste” in the U.S., as “Municipal solid waste” (residential) or “Commercial & Industrial Waste” (commercial/industrial) in the U.K., or colloquially as garbage/refuse/trash/etc. The aforementioned waste types are referred to here as “MSW”. The MSW can include agricultural waste. “Waste” generally refers to carbon-containing combustible material that has been discarded after its primary use, including solid waste. Generally, the waste may be wet and heterogeneous, containing a portion of non-combustible waste. “Solid waste” refers to any garbage or refuse, sludge from a wastewater treatment plant, water supply treatment plant, or air pollution control facility and other discarded material, including solid, liquid, semi-solid, or contained gaseous material resulting from industrial, commercial, mining, and agricultural operations, and from community activities.

A variety of sources of solid waste can be used. The solid waste mixture may be derived from waste sources (e.g., non-hazardous waste sources) including, but not limited to, municipal waste, agricultural waste, sewage sludge, household waste, discarded secondary materials, and industrial solid waste. “Municipal waste,” or “municipal solid waste” (MSW), as used herein, may refer to any household waste or commercial solid waste or industrial solid waste. Non-limiting examples of wastes that may be included in the solid waste mixture include biodegradable waste such as food and kitchen waste; green wastes such as lawn or hedge trimmings; paper; mixed plastics; solid food waste; solid agricultural waste; sewage sludge; and automotive shredder residue.

“Household waste” or “residential waste” refers to any solid waste (including garbage, trash, and sanitary waste in septic tanks) derived from households (including single and multiple residences, hotels and motels, bunkhouses, ranger stations, crew quarters, campgrounds, picnic grounds, and day-use recreation areas).

“Commercial solid waste” refers to all types of solid waste generated by stores, offices, restaurants, warehouses, and other nonmanufacturing activities, excluding residential and industrial wastes.

“Industrial solid waste” refers to solid waste (e.g., non-hazardous solid waste) generated by manufacture or industrial processes. Examples of industrial solid waste include, but are not limited to, waste resulting from the following manufacturing processes: Electric power generation; fertilizer/agricultural chemicals; food and related products/by-products; leather and leather products; organic chemicals; plastics and resins manufacturing; pulp and paper industry; rubber and miscellaneous plastic products; textile manufacturing; transportation equipment; and water treatment. This term does not include mining waste or oil and gas waste.

The solid waste mixture may comprise discarded non-hazardous secondary material, in which case solid fuel compositions produced from those solid waste mixtures may be legally categorized as “non-waste.” “Secondary material” refers to any material that is not the primary product of a manufacturing or commercial process, and can include post-consumer material, off-specification commercial chemical products or manufacturing chemical intermediates, post-industrial material, and scrap. Examples of non-hazardous secondary materials include scrap tires that are not discarded and are managed by an established tire collection program, including tires removed from vehicles and off-specification tires; resinated wood; coal refuse that has been recovered from legacy piles and processed in the same manner as currently-generated coal refuse; and dewatered pulp and paper sludges that are not discarded and are generated and burned on-site by pulp and paper mills that burn a significant portion of such materials where such dewatered residuals are managed in a manner that preserves the meaningful heating value of the materials.

“Resinated wood” refers to wood products (containing binders and adhesives) produced by primary and secondary wood products manufacturing. Resinated wood includes residues from the manufacture and use of resinated wood, including materials such as board trim, sander dust, panel trim, and off-specification resinated wood products that do not meet a manufacturing quality or standard.

The MSW can be a mixed solid waste and may have a highly variable composition due to the variable nature of municipal solid waste streams. A municipal solid waste stream may vary in composition due to a variety of factors including, but not limited to, different seasons, different locations within a country (urban versus rural), and/or different countries (industrial versus emerging).

In some embodiments, the MSW is a non-hazardous solid waste. In some embodiments, the MSW is substantially free of medical waste. “Medical waste” (also referred as biomedical waste or hospital waste) refers to waste that contains infectious or potentially infectious material and is typically regulated by state and/or federal agencies. Medical waste is distinct from common waste, such as municipal solid waste, commercial waste, household/residential waste, and industrial waste. Medical waste may also include waste associated with the generation of biomedical waste that visually appears to be of medical or laboratory origin (e.g., packaging, unused bandages, infusion kits), as well as research laboratory waste containing biomolecules and/or organisms that are mainly restricted from environmental release. Medical waste can include solids, liquids, or both. Some non-limiting examples of medical waste include pathological waste, human blood and blood products, cultures and stocks of infectious agents, contaminated sharps, isolation waste, and contaminated animal carcasses, body parts, and bedding (e.g., intentionally exposed to pathogens in research, biologicals productions, or in vivo testing). In some embodiments, the MSW is substantially free of medical waste (e.g., regulated medical waste).

The disclosed facility includes a “contaminated” zone and a “clean” zone. The facility can be a new facility or a retro-fitted existing waste transfer station, materials recovery facility, or recycling center. Waste collection vehicles (such as garbage trucks) carry MSW into the contaminated zone. The subject matter described can be implemented in particular embodiments, so as to realize one or more of the following advantages. In some embodiments, the waste collection vehicles are decontaminated upon leaving the facility, for example, by electrostatic spray, so that they return to communities in a decontaminated state. Within the contaminated zone, the MSW can be sorted and/or shredded. Within the contaminated zone, the MSW is transported to thermal treatment system(s), where the MSW is sanitized and/or sterilized. The processed MSW is transported to the clean zone. Within the clean zone, the processed MSW can be cooled. The processed MSW can be transported to a storage bunker, a hopper, or a vehicle. In some cases, the MSW is thermally treated to remove moisture, sanitize the material, and/or begin thermolysis (at certain temperatures) of some or all of the biological material contained in the MSW. Such thermally processed MSW can be densified. Densification can result in weight reduction (e.g., through moisture removal) and volume reduction. Therefore, even if brought to a landfill, the densified, processed MSW can lengthen the life of the landfill due to the reduced volume. Further, the processed MSW can mitigate transmission of diseases, for example, by avian means because the MSW has been converted into an unattractive food source. Moreover, the thermal treatment of the MSW can prevent infectious agents from being introduced to a landfill. Alternatively, the processed MSW can be used, for example, as a fuel (due to its heat content) or as a raw material for a reuse or recycled product.

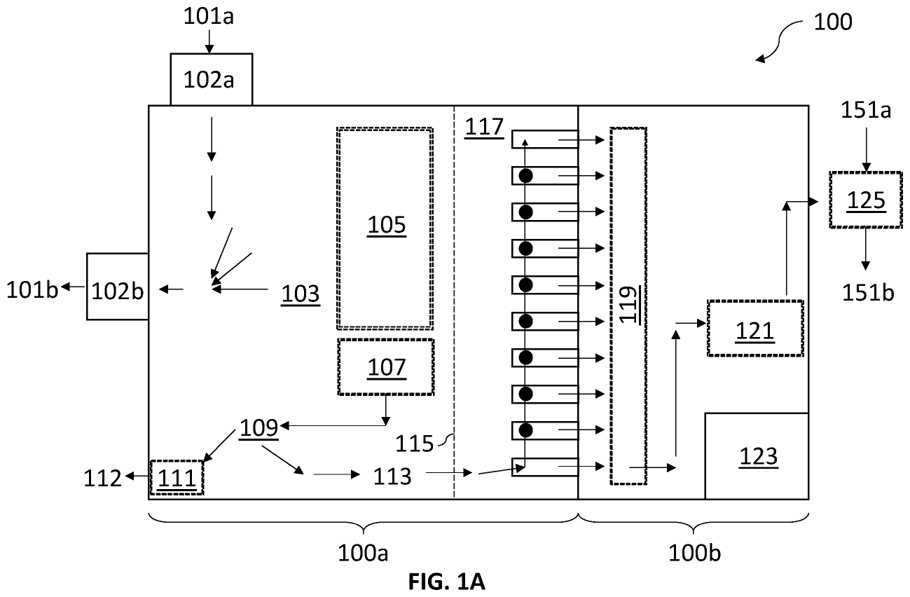

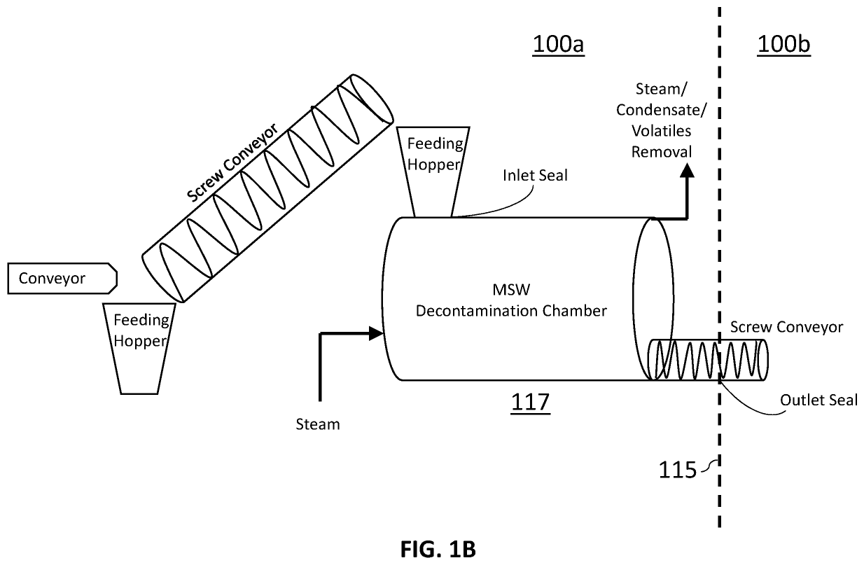

FIG. 1A depicts a solid waste treatment facility 100. Existing waste collection vehicles (e.g., garbage trucks) can be used. The layout balances requirements of an isolation room with the mechanics of how a garbage truck operates and maneuvers, with cumbersome maneuvering minimized. The truck(s) 101a enter the facility 100 through an intermediate containment room 102a, then exit and enter isolation zone 100a (contaminated zone). In some embodiments, the isolation zone 100a has a negative air pressure relative to an external environment. “External environment” refers to any area external to the area of interest, such as the isolation zone 100a. For example, the external environment is an area or simply the air surrounding the isolation zone 100a. With respect to FIG. 1A, the vehicle 101a can drive straight into the isolation zone 100a, hit reverse and turn wheel for 90 degree, dump waste, then drive straight out of the isolation zone 100a. This process can be achieved in a few minutes per vehicle. The facility 100 can include an enclosed structure. The walls of the enclosed structure can, for example, define the boundary limits of the facility 100, and the isolation zone 100a and the clean zone 100b can be distinct areas within the enclosed structure. The isolation zone 100a and the clean zone 100b are separated by a barrier (e.g., by a halfwall 115) that prevents cross-contamination between the zones 100a, 100b. The barrier can include a wall (e.g., the halfwall 115), a curtain (e.g., an industrial air curtain), a door, a window, or any combination of these.

In some embodiments, at least one waste collection vehicle (for example, one, two, three, or more than three waste collection vehicles) can fit within the isolation zone 100a and unload waste in the isolation zone 100a. In some cases, multiple waste collection vehicles can fit within the isolation zone 100a at the same time and unload waste in the isolation zone 100a simultaneously. The waste collection vehicle can be any transport vehicle that includes a compartment for receiving and transporting waste. Some non-limiting examples of waste collection vehicles include a dump truck, a garbage truck, a refuse vehicle, a semi-truck, and a dustbin lorry. The waste collection vehicles can be front loaders, rear loaders, automated side loaders, pneumatic collection vehicles, and/or grapple trucks.

The truck(s) 101a drop off waste (for example, MSW) in the unloading zone 103. In some embodiments, the facility 100 includes a tipping floor 105, a push floor 107, or a combination of these. The tipping floor 105 can include an enclosed floor having a surface onto which waste is deposited from a collection container or vehicle. The tipping floor 105 can be used to stage waste for reloading into processing equipment, for reloading into transport vehicle(s), and/or for removal of recyclable materials. In some embodiments, a tipping floor 105 includes container(s) and/or truck(s) for consolidating waste for future transport. The push floor 107 can be used to transport waste from the tipping floor 105 to a conveyor without direct human operator contact with the waste. For example, the push floor 107 is a hydraulic push floor that enables minimal or no contact between human personnel and the waste. In some embodiments, the waste may be dropped onto the push floor 107 or otherwise onto a raised structure intended to capture waste (such as a hopper), thereby mechanically loading the waste onto a conveyor. In some embodiments, the push floor 107 includes one or more metal frames placed against a floor (which can have a square shape, rectangular shape, or any other typical floor shape), and the one or more metal frames can slide back and forth, thereby transporting the material, for example, towards a conveyor. The conveyor can be a screw conveyor or a trough chain conveyor. After dropping off the waste, the empty truck(s) 101b exits the facility 100 through an intermediate containment room 102b or sealed area. An industrial air curtain may be positioned over each door to and from containment areas 102a, 102b (for example, at each entry and exit).

The facility 100 can include a ripping drum, a shredding bag opener, or both in zone 100a. The waste can be sorted in sorting zone 109. For example, recyclable material 112 can be sorted out from the waste in sorting zone 109. For example, metal (such as ferrous and non-ferrous metal), glass, aggregates (such as rocks, sand, and dirt), and plastics (such as PVC) can be sorted out from the waste in the sorting zone 109. The recyclable material 112 can be compressed by a baler 111 and exit the facility 100. In some embodiments, materials sorted out from the waste (for example, metals, glass, aggregates, and plastics) are sprayed with an electrostatic spray and/or washed with a chemical agent to decontaminate the metals. In some embodiments, materials sorted out from the waste are stored (for example, outside of the zone 100a but still on the premises of the facility 100) for a length of time sufficient for pathogens of concern to become non-viable before they are transported out of the facility 100. In some embodiments, materials sorted out from the waste are placed into containers and baled for recycling. The materials sorted out from the waste can be further sorted. In some embodiments, a magnet is used to sort out ferrous metals. In some embodiments, an eddy current sorting machine is used to sort out non-ferrous metals. In some embodiments, an air classifier is used to sort out glass and dirt. In some embodiments, a trammel is used to sort out fines, such as sand or dirt. For example, an optical sorter can be used to identify and sort out plastics, such as PVC.

The sorted waste can be shredded in shredding zone 113. FIG. 1B shows a portion of the facility 100 that handles transporting the waste to the thermal treatment system(s) 117 and transporting the processed waste to a post-treatment zone 100b. After being sorted in zone 109 and/or shredded in zone 113 within the isolation zone 100a, the waste can travel on a conveyor (e.g., an enclosed conveyor) through a halfwall 115 onto a raised distribution system. The waste travels through a treatment systems area 117 that includes treatment system(s). The waste can travel on a conveyor above the treatment systems and into each chamber of the treatment systems. In some embodiments, the waste drops from the conveyor into a small hopper (e.g., one per treatment system) that is attached to the treatment system by a bulk material feeding valve (e.g., a rotary airlock valve). Once the waste is transported into the treatment system(s), the inlet opening(s) are closed and sealed.

The treatment system(s) can be configured to heat the waste. In some embodiments, the treatment systems are configured to heat a majority of the waste to a temperature of at least 121° C., at least 132° C., at least 141° C., or at least 160° C. In some embodiments, the treatment systems are configured to maintain a majority of the waste at a temperature of at least 121° C., at least 132° C., at least 141° C., or at least 160° C. for a length of time. The length of time can be at least 4 minutes, at least 20 minutes, or at least 30 minutes.

The treatment system(s) can be configured to operate at negative pressure (vacuum), atmospheric pressure, positive pressure, or a combination of these at different times. For example, steam can be introduced to the treatment system(s) for a sufficient length of time to sanitize and/or sterilize the waste. The steam can be at atmospheric pressure or at a pressure greater than atmospheric pressure (for example, pressurized steam). For example, the steam has an operating pressure of at least 0 psig, at least 1 psig, at least 5 psig, at least 10 psig, or at least 15 psig. The operating temperature of the steam can be at least 121° C., at least 132° C., at least 141° C., or at least 160° C. Using pressurized steam to sanitize and/or sterilize the waste may be advantageous to using steam at atmospheric pressure. For example, in some cases, pressurized steam operates at a greater temperature in comparison to steam at atmospheric pressure. For example, pressurized steam may penetrate into the waste more quickly than steam at atmospheric pressure. For example, pressurized steam may heat the waste more quickly in comparison to steam at atmospheric pressure. In some embodiments, pressurized steam is introduced to the treatment system(s), such that the waste in the treatment system(s) is maintained at a temperature of at least 121° C., at least 132° C., at least 141° C., or at least 160° C. for a length of time. The length of time can be at least 4 minutes, at least 20 minutes, or at least 30 minutes. In some embodiments, a vacuum (partial or full) is pulled on the treatment system(s) to evacuate vapor (such as volatile compounds and steam) from the treatment system(s). Exposing the waste to negative pressure can dehydrate the waste, which can be advantageous, especially in cases where the processed waste is to be used as fuel. In some embodiments, the vacuum is pulled after the waste is sanitized and/or sterilized (for example, by introduction of the pressurized steam). Vacuum can be pulled in the chamber(s) of the thermal treatment system(s), for example, by using a vacuum pump. In some embodiments, a majority of emissions produced by the thermal treatment system(s) is water vapor. For example, the emissions produced by the thermal treatment system(s) include at least about 50%, 60%, 70%, 80%, 90%, 95%, 96%, 97%, 98%, 99%, 99.5%, 99.9%, 99.99%, or at least about 99.999% or more (by weight) of water.

In some embodiments, the thermal treatment system(s) include agitator(s) that can agitate the waste. For example, the agitator(s) can agitate the waste during the sanitization/sterilization process and/or during the vapor evacuation process. Agitation may decrease the processing time necessary to sanitize, sterilize, and/or dehydrate the waste in the treatment system(s). In some embodiments, the agitator(s) provide heat to the waste. For example, the agitator(s) can be configured to provide heat to the waste, such that the waste is brought to and maintained at a temperature of at least 121° C., at least 132° C., at least 141° C., or at least 160° C. for a length of time, which can be at least 4 minutes, at least 20 minutes, or at least 30 minutes. For example, a heating fluid (such as hot oil) is flowed through the agitator(s) to provide heat to the waste. For example, the agitator(s) can be electrically heated to provide heat to the waste. In some embodiments, the thermal treatment system(s) include heating jacket(s) that can provide heat to the waste. For example, a heating fluid (such as hot oil) can be flowed through the heating jacket(s) to provide heat to the waste. In contrast to the agitator(s), the heating jacket(s) can be stationary. The heating jacket(s) can be disposed within the chamber(s) of the thermal treatment system(s) to be put in direct contact with the waste to provide conductive heat to the waste or to provide convective (non-conductive) heat to the waste. The heating jacket(s) can be disposed around the chamber(s) of the thermal treatment system(s). In some embodiments, the thermal treatment system(s) include electrical heater(s) that can provide heat to the waste. For example, electricity is provided to the electrical heater(s), which convert the electricity into heat that is provided to the waste. The electrical heater(s) can be stationary (similar to the heating jacket), or the agitator(s) can include electrical heater(s), in which case the electrical heater(s) move with the agitator(s). Heat can be provided to the waste by the agitator(s), by the electric heater(s), by the heating jacket(s), by introducing steam to the thermal treatment system(s), or a combination of these.

If the thermal treatment system(s) operate at negative pressure, then when the treated material is passing through the wall, the negative pressure will keep the air flow in a reverse direction, e.g., back to the isolation zone 100a, thereby keeping the post-treatment zone 100b clean. If the thermal treatment system(s) operate at positive pressure or atmospheric pressure, then the inlet portion of the treatment system can be sealed such that when material exits it and goes into post-treatment zone 100b, there is no open connection between isolation zone 100a and post-treatment zone 100b.

In some embodiments, the thermal treatment system(s) are configured to provide heat to the waste, such that the waste is brought to and maintained at a temperature at which sanitation and/or sterilization of the waste occurs but incineration of the waste does not occur. Incineration involves a combustion reaction in which a fuel (e.g., waste) and an oxidant (e.g., oxygen) react to produce oxidized (typically gaseous) products, such as water vapor and/or carbon dioxide. In some embodiments, the thermal treatment system(s) are configured to provide heat to the waste, such that the waste is brought to and maintained at a temperature at which sanitation and/or sterilization of the waste occurs but pyrolysis and/or gasification of the waste does not occur. Pyrolysis is a type of thermolysis, resulting in the irreversible thermal decomposition of organic material (e.g., MSW). Pyrolysis involves a change in chemical composition and physical phase in the absence of oxygen, where feedstock is changed into ash, char (such as biochar), synoil (biooil), and syngas (biogas). Gasification is similar to pyrolysis in that it involves thermal decomposition of volatile components in an organic substance (e.g., MSW), but also involves conversion of non-volatile carbon char to produce syngas in the presence of a small amount of air/oxygen or in the absence of oxygen. Gasification typically occurs at temperature ranges greater than those associated with pyrolysis and also typically produces more syngas in comparison to pyrolysis. For example, pyrolysis can occur at temperatures in a range of from about 200° C. to about 760° C., and gasification can occur at temperatures in a range of from about 480° C. to about 1650° C. In some embodiments, the thermal treatment system(s) are configured to sanitize and/or sterilize the waste independent of autoclaving.

The thermal treatment system(s) can be configured to dry the waste. Drying the waste can involve heating the waste, exposing the waste to a negative pressure, agitating the waste, or any combination of these. In some embodiments, the thermal treatment systems are configured to dry the waste to yield a dried waste having a water content in a range of from 0.1% to 10%, less than 10%, 9%, 8%, 7%, 6%, 5%, 4%, 3%, 2%, 1%, 0.5%, or less than 0.1% (by weight). In some embodiments, the waste is dried before it is heated to and maintained at a temperature sufficient for sanitizing and/or sterilizing the waste.

In some embodiments, the treated material is extruded. For example, the treated material can be extruded from the chamber(s) of the thermal treatment system(s). Once the waste inside the treatment system(s) is sanitized and/or sterilized, the waste exits the chamber(s) of the treatment system(s) and can be moved into the post-treatment zone 100b, which is designated as a clean zone with a separate HVAC system. An outlet point of each treatment system can pass through a wall (such as the halfwall 115) which acts as a barrier between zones 100a and 100b. In some embodiments, the halfwall 115 includes an industrial air curtain.

Referring back to FIG. 1A, the treated material can be cooled by a cooling system 119 before the treated material exits the facility 100 given its high temperature (include to limit oxygen exposure). The cooling system 119 is configured to cool the treated material. In some embodiments, the cooling system 119 is an air cooling system. The cooling system 119 can include a fan that circulates air to cool the treated material. In some embodiments, letting the treated waste to sit in the cooling zone 121 (for example, with or without cooling by the cooling system 119) for 24 hours typically allows a sufficient reduction in temperature.

The processed waste material can be removed as a bulk material and transported out of the facility 100, for example, into a raised hopper 125. Empty truck(s) 151a can drive under the hopper 125 and be automatically loaded with the processed waste material. Truck(s) 151b loaded with the processed waste material can transport the processed waste material to be used, for example, as a fuel. In some embodiments, the post-treatment zone 100b includes one or more offices 123. In some embodiments, the one or more offices 123 share a common HVAC system

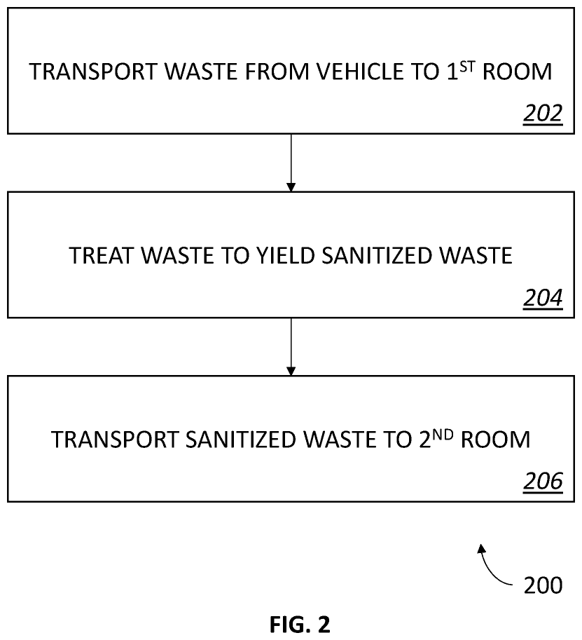

FIG. 2 is a flow chart of a method 200 of treating solid waste (such as MSW) at a waste treatment facility (such as the facility 100). At step 202, waste is transported from a waste collection vehicle (such as the truck(s) 101a) to a first room (or a sealed area) of a facility, such as the contaminated zone 100a of the facility 100. At step 204, the waste is treated in the first room (contaminated zone 100a) to destroy a majority of pathogens in the waste to yield a sanitized waste. At step 206, the sanitized waste is transported to a second room (or a second sealed area) of the facility, such as the clean zone 100b of the facility 100. Throughout the method 200, pathogens in the waste can be prevented from entering the clean zone 100b.

In some embodiments, the waste is sorted and/or shredded before it is treated at step 204. For example, the waste can be unloaded from the truck(s) 101a and sorted in sorting zone 109. For example, sorting the waste can include removing recyclables from the waste and baling the recyclables.

Treating the waste at step 204 can include heating the waste, exposing the waste to positive pressure (that is, pressure greater than atmospheric pressure), exposing the waste to negative pressure (that is, pressure less than atmospheric pressure), extruding the waste, or any combination of these. In some embodiments, a majority of the waste is heated to a temperature of at least 121° C., at least 132° C., at least 141° C., or at least 160° C. In some embodiments, a majority of the waste is heated for at least 4 minutes, at least 20 minutes, or at least 30 minutes. In some embodiments, the waste is heated at step 204, such that a majority of the waste is maintained at a temperature of at least 121° C., at least 132° C., at least 141° C., or at least 160° C. for at least 4 minutes, at least 20 minutes, or at least 30 minutes. Heating the waste can include electrical heating, conductive heating, convective heating, introducing steam to the waste, or any combination of these. In some embodiments, the waste is exposed to positive pressure before being heated, while being heated, after being heated, or any combination of these. In some embodiments, the waste is exposed to negative pressure before being heated, while being heated, after being heated, or any combination of these. In some embodiments, the waste is exposed to a variety of pressures during the waste treatment at step 204. For example, the waste is exposed to a positive pressure while heating, and then the waste is exposed to a negative pressure after heating. In some embodiments, the waste is exposed to a negative pressure throughout the waste treatment at step 204. In some embodiments, treating the waste at step 204 includes agitating the waste. For example, the waste can be agitated while the waste is being heated, while the waste is exposed to positive pressure, while the waste is exposed to negative pressure, or any combination of these. In some embodiments, treating the waste at step 204 includes extruding the waste. For example, the waste is extruded after being exposed to negative pressure. Treating the waste at step 204 can yield a fuel composition, which can, for example, be burned. Treating the waste at step 204 can yield a raw material for a reuse or recycled product.

In some cases, the waste includes residual MSW (“rMSW”) which can include food waste, diapers, bandages, plastic (for example, in the form of plastic film), paper, yard waste, animal carcass, animal feces, or a combination of these. The rMSW can be shredded or otherwise reduced into smaller sizes. For example, the rMSW can be reduced, such that a majority of the rMSW is in the form of discrete parts having a maximum dimension less than 2 inches, less than 5 inches, less than 10 inches, less than 20 inches, in a range of from 2 inches to 20 inches, in a range of from 2 inches to 10 inches, or in a range of from 2 inches to 5 inches. The rMSW can be transported for further decontamination (for example, to the thermal treatment system(s)).

As used in this disclosure, the terms “sterilization” and “sanitization” means inactivation and/or destruction of microbes, which can include pathogens.

As used in this disclosure, the phrase “a majority of” means at least about 50%, 60%, 70%, 80%, 90%, 95%, 96%, 97%, 98%, 99%, 99.5%, 99.9%, 99.99%, or at least about 99.999% or more (by weight).

As used in this disclosure, the term “about” or “approximately” can allow for a degree of variability in a value or range, for example, within 10%, within 5%, or within 1% of a stated value or of a stated limit of a range.

As used in this disclosure, the term “substantially” refers to a majority of, or mostly, as in at least about 50%, 60%, 70%, 80%, 90%, 95%, 96%, 97%, 98%, 99%, 99.5%, 99.9%, 99.99%, or at least about 99.999% or more (by weight).

As used in this disclosure, the phrase “substantially free of” means having less than about 10%, 9%, 8%, 7%, 6%, 5%, 4%, 3%, 2%, 1%, 0.5%, 0.1%, 0.01%, or less than about 0.001% of (by weight).

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A solid waste treatment facility comprising:
a first room defining an isolation zone having at least one access gate, the at least one access gate sized to receive a waste collection vehicle, the first room being an enclosed area and configured to receive mixed solid waste from the waste collection vehicle, the first room comprising:
a receiving area configured for receiving the mixed solid waste from the waste collection vehicle; and
at least one process vessel configured to reduce the amount of or eliminate at least a portion of pathogens present in the mixed solid waste to yield sanitized waste that is free of or substantially free of pathogens; and
a second room defining a clean zone, the second room separated from the first room by a barrier, the second room configured to receive the sanitized waste from the first room.

2. The facility of claim 1, wherein the mixed solid waste comprises municipal waste obtained from a residential source, an institutional source, a commercial source, an agricultural source, a sewage source, or combinations thereof.

3. The facility of claim 1, wherein the mixed solid waste is substantially free of medical waste originating from a laboratory or dedicated building that provides medical care.

4. The facility of claim 1, wherein the first room comprises one or more containment areas, and the one or more containment areas are configured to receive the waste collection vehicle.

5. The facility of claim 1, comprising a heater operatively coupled to the at least one process vessel, wherein the at least one process vessel is configured to receive an amount of the mixed solid waste and increase a temperature of the mixed solid waste to yield the sanitized waste that is free of or substantially free of pathogens.

6. The facility of claim 1, wherein the first room defines a negative air pressure relative to an external environment.

7. The facility of claim 1, wherein the first room comprises one or more hydraulic push floors, tipping floors, or combinations thereof, configured to move the mixed solid waste from the receiving area to the at least one process vessel, move the sanitized waste from the first room to the second room, or both.

8. The facility of claim 1, wherein the second room comprises a cooling system configured to cool sanitized waste, and the cooling system is an air cooling system.

9. A method of treating solid waste at a waste treatment facility, the method comprising:
transporting mixed solid waste from a waste collection vehicle to a first room of the facility, the first room defining an enclosed isolation zone configured to receive the mixed solid waste from the waste collection vehicle;
treating the mixed solid waste in at least one process vessel in the first room to destroy a majority of pathogens in the mixed solid waste to yield a sanitized waste; and
transporting the sanitized waste to a second room of the facility, the second room defining a clean zone separated from the first room by a barrier.

10. The method of claim 9, comprising sealing the isolation zone of the first room by applying a negative air pressure relative to an external environment outside the first room and/or the facility.

11. The method of claim 9, wherein treating the mixed solid waste comprises heating a majority of the mixed solid waste to a temperature of at least 121° C., at least 132° C., at least 141° C., or at least 160° C.

12. The method of claim 11, wherein heating the mixed solid waste comprises heating a majority of the mixed solid waste to at least 121° C. for at least 30 minutes or at least 132° C. for at least 4 minutes.

13. The method of claim 9, wherein treating the mixed solid waste comprises drying the mixed solid waste to yield a dried waste comprising about 5 wt. % or less of water.

14. The method of claim 9, wherein treating the mixed solid waste comprises exposing the mixed solid waste or the dried waste to negative pressure.

15. The method of claim 9, wherein treating the mixed solid waste comprises exposing the mixed solid waste or the dried waste to an operating pressure of atmospheric pressure or greater and agitating the mixed solid waste during the heating.

16. The method of claim 9, wherein transporting the mixed solid waste from the waste collection vehicle comprises moving the mixed solid waste, in an unsealed state, onto one or more tipping floors, hydraulic push floors, or combinations thereof, and feeding the mixed solid waste to the at least one process vessel via a conveyor.

17. The method of claim 9, comprising treating the mixed solid waste to yield a waste product for landfill allocation or a raw material for a reuse or recycled product.

18. The method of claim 9, comprising preventing pathogens in the mixed solid waste from entering the second room of the facility by applying positive pressure in the second room, separating the first and second rooms with a barrier, or combinations thereof.

19. The method of claim 9, wherein the method does not incinerate or autoclave the mixed solid waste to yield the sanitized waste.

20. A method of treating solid waste at a waste treatment facility, the method comprising:
transporting mixed solid waste from a waste collection vehicle to a first room of the facility, the first room defining an enclosed isolation zone configured to receive the mixed solid waste from the waste collection vehicle, the mixed solid waste being substantially free of medical waste;
treating the mixed solid waste in at least one process vessel in the first room to destroy a majority of pathogens in the mixed solid waste to yield a sanitized waste; and
transporting the sanitized waste to a second room of the facility, the second room defining a clean zone separated from the first room by a barrier.
